SEQUENCE LISTING (28 sequences)

SEQ ID NO: 1 - DNA, 18 bp, synthetic construct
  source 1..18: mol_type = other DNA
cagagcattggtagtaat

SEQ ID NO: 2 - DNA, 36 bp, synthetic construct
  source 1..36: mol_type = other DNA
caacagggttatagtgataattatcttgataatgct

SEQ ID NO: 3 - protein, 6 aa, synthetic construct
QSIGSN

SEQ ID NO: 4 - protein, 12 aa, synthetic construct
QQGYSDNYLDNA

SEQ ID NO: 5 - DNA, 21 bp, synthetic construct
  source 1..21: mol_type = other DNA
cagagtattagtagtagctac

SEQ ID NO: 6 - DNA, 36 bp, synthetic construct
  source 1..36: mol_type = other DNA
caatatagtgattgggctaatagttatgggaatgct

SEQ ID NO: 7 - protein, 7 aa, synthetic construct
QSISSSY

SEQ ID NO: 8 - protein, 12 aa, synthetic construct
QYSDWANSYGNA

SEQ ID NO: 9 - DNA, 27 bp, synthetic construct
  source 1..27: mol_type = other DNA
ggaatcgacttcagtgccggctactac

SEQ ID NO: 10 - DNA, 27 bp, synthetic construct
  source 1..27: mol_type = other DNA
atttatgctggtcgtagtcttaacact

SEQ ID NO: 11 - DNA, 39 bp, synthetic construct
  source 1..39: mol_type = other DNA
gcgagagatggggctagcagtggctactactttaagttg

SEQ ID NO: 12 - protein, 9 aa, synthetic construct
GIDFSAGYY

SEQ ID NO: 13 - protein, 9 aa, synthetic construct
IYAGRSLNT

SEQ ID NO: 14 - protein, 13 aa, synthetic construct
ARDGASSGYYFKL

SEQ ID NO: 15 - DNA, 27 bp, synthetic construct
  source 1..27: mol_type = other DNA
ggattctccttcagtagcggctactac

SEQ ID NO: 16 - DNA, 24 bp, synthetic construct
  source 1..24: mol_type = other DNA
atttatggtggtaataataaccca

SEQ ID NO: 17 - DNA, 36 bp, synthetic construct
  source 1..36: mol_type = other DNA
agaaaagatattaatattggtggtgcttatgagttg

SEQ ID NO: 18 - protein, 9 aa, synthetic construct
GFSFSSGYY

SEQ ID NO: 19 - protein, 8 aa, synthetic construct
IYGGNNNP

SEQ ID NO: 20 - protein, 12 aa, synthetic construct
RKDINIGGAYEL

SEQ ID NO: 21 - DNA, 363 bp, synthetic construct
  source 1..363: mol_type = other DNA
gaggtccagctgctggagagcggaggaggactggtgcagcccggaggatctttaagactgagctgtgccgccagcggcttcagcttcagcagcggctactacatctgctgggtgagacaagctcccggtaaaggtttagagtggatcgcttgtatctacggcggcaacaacaacccctactacgccaactgggtgaacggcagattcaccatctctcgtgacaacagcaagaacactttatatttacagatgaactctttaagggccgaggacaccgccgtgtactactgcgctcgtaaggacatcaacatcggcggcgcctatgagctgtggggccaaggtactttagtgaccgtgagcagc

SEQ ID NO: 22 - DNA, 333 bp, synthetic construct
  source 1..333: mol_type = other DNA
gacatccagatgacccagagccctagctctttaagcgccagcgtgggcgacagagtgaccatcacttgtcaagctagccagagcatcagcagcagctatttaagctggtaccagcagaagcccggcaaggcccccaagctgctgatctacaaggccagcacactggccagcggcgtgccttctcgttttagcggcagcggcagcggaaccgacttcactttaaccatcagctctttacagcccgaggacttcgccacctactactgccagtacagcgactgggccaacagctatggcaacgccttcggcggcggcaccaaggtggagatcaag

SEQ ID NO: 23 - protein, 121 aa, synthetic construct
EVQLLESGGGLVQPGGSLRLSCAASGFSFSSGYYICWVRQAPGKGLEWIACIYGGNNNPYYANWVNGRFTISRDNSKNTLYLQMNSLRAEDTAVYYCARKDINIGGAYELWGQGTLVTVSS

SEQ ID NO: 24 - protein, 111 aa, synthetic construct
DIQMTQSPSSLSASVGDRVTITCQASQSISSSYLSWYQQKPGKAPKLLIYKASTLASGVPSRFSGSGSGTDFTLTISSLQPEDFATYYCQYSDWANSYGNAFGGGTKVEIK

SEQ ID NO: 25 - DNA, 360 bp, synthetic construct
  source 1..360: mol_type = other DNA
cagtcggtgaaggagtccgggggagacctggtcaagcctggggcttccctgacactcacctgcacagcctctggaatcgacttcagtgccggctactacatgtgctgggtccgccaggctccagggaaggggctggagttgatcgcatgcatttatgctggtcgtagtcttaacactttctacgcgagctgggcgaaaggccgattcaccatctccagagcctcgtcgaccacggtgactctggcgatgaccagtctgacagccgcggacacggccacctatttctgtgcgagagatggggctagcagtggctactactttaagttgtggggcccaggcaccctggtcaccatctcttca

SEQ ID NO: 26 - DNA, 330 bp, synthetic construct
  source 1..330: mol_type = other DNA
gagctcgatatgacccagactccagcctctgtggaggtagctgtgggaggcacagtcaccatcaagtgccaggccagtcagagcattggtagtaatttagcctggtatcagcagaaaccagggcagcgtcccaatgtcctgatctacgatgcatcgaatctggcatctggggtctcatcgcggttcaaaggcagtggatctgggacacagttcactctcaccatcagcggcggggagtgtgccgatgctgccacttactactgtcaacagggttatagtgataattatcttgataatgctttcggcggagggaccgaggtggtggtcaaa

SEQ ID NO: 27 - protein, 120 aa, synthetic construct
QSVKESGGDLVKPGASLTLTCTASGIDFSAGYYMCWVRQAPGKGLELIACIYAGRSLNTFYASWAKGRFTISRASSTTVTLAMTSLTAADTATYFCARDGASSGYYFKLWGPGTLVTISS

SEQ ID NO: 28 - protein, 110 aa, synthetic construct
ELDMTQTPASVEVAVGGTVTIKCQASQSIGSNLAWYQQKPGQRPNVLIYDASNLASGVSSRFKGSGSGTQFTLTISGGECADAATYYCQQGYSDNYLDNAFGGGTEVVVK

(19) United States
(12) Patent Application Publication
(10) Pub. No.: US 2026/0118369 A1
(43) Pub. Date: Apr. 30, 2026

(54) ASSAYS FOR DETECTION AND QUANTITATION OF HUMAN ENDOTROPHIN

(71) Applicant: THE BOARD OF REGENTS OF THE UNIVERSITY OF TEXAS SYSTEM, Austin, TX (US)

(72) Inventors: Ningyan ZHANG, Houston, TX (US); Zhiqiang AN, Houston, TX (US); Philipp SCHERER, Dallas, TX (US); Wei XIONG, Houston, TX (US)

(73) Assignee: THE BOARD OF REGENTS OF THE UNIVERSITY OF TEXAS SYSTEM, Austin, TX (US)

(21) Appl. No.: 18/998,277

(22) PCT Filed: Jul. 27, 2023
(86) PCT No.: PCT/US2023/071084

§ 371 Date: Jan. 24, 2025

Related U.S. Application Data
(60) Provisional application No. 63/393,068, filed on Jul. 28, 2022.

(51) Int. Cl. G01N 33/68

(52) U.S. Cl. CPC G01N 33/6887; G01N 2333/78

(57) ABSTRACT

The present disclosure is directed to antibodies binding to human endotrophin and methods for use of such antibodies to detect endotrophin in human samples.

### PRIORITY CLAIM

This application is a national phase application under 35 U.S.C. § 371 of International Application No. PCT/US2023/071084, filed Jul. 27, 2023, which claims benefit of priority to U.S. Provisional Application Ser. No. 63/393,068 filed Jul. 28, 2022, the entire contents of each of which are hereby incorporated by reference.

### REFERENCE TO A SEQUENCE LISTING

This application contains a Sequence Listing XML, which has been submitted electronically and is hereby incorporated by reference in its entirety. Said XML Sequence Listing, created on Jul. 19, 2023, is named UTFHP0391WO.xml and is −27 kilobytes in size.

### BACKGROUND

### 1. Field of the Disclosure

The present disclosure relates generally to the fields of medicine, pathology, and immunology. More particularly, the disclosure relates to assays to detect and quantitate human endotrophin.

### 2. Background

Collagen VI (ColVI) is a type of collagen primarily associated with the extracellular matrix of skeletal muscle. ColVI is also found in the skin, lungs, blood vessels, cornea and intervertebral disc. It also forms part of the peripheral nerves, brain, myocardium and adipose tissue. ColVI molecules are made up of three alpha chains: α1(VI), α2(VI), and α3(VI). It is encoded by 6 genes: COL6A1, COL6A2, COL6A3, COL6A4, COL6A5, and COL6A6. The chain lengths of α1(VI) and α2(VI) are about 1,000 amino acids. The chain length of α3(VI) is roughly a third larger than those of α1(VI) and α2(VI), and it consists of several spliced variants within the range of 2,500 to 3,100 amino acids.

Collagen VI plays many different roles in the cell depending on which tissue in which it is expressed. ColVI maintains a mechanical function in the cell, which is typical of most types of Collagen, by providing stability and structural support in the ECM. ColVI allows muscle cells to connect with the ECM by interacting with perlecan in the basal lamina. ColVI also functions as a cytoprotective agent:
plays an important role in cancer by acting as a chemotherapy resistance modulator; inhibits oxidative damage and apoptosis;
regulates cell differentiation and autophagic machinery;
with the assistance of other collagens, proteoglycans, matrilineal, fibronectins, and glycoproteins, anchors the basement membrane of the skin to the extracellular matrix
ColVI plays a key role in the extracellular matrix of white adipose tissue. Lack of ColVI in the extracellular matrix of white adipose tissue leads to molecular characteristic notably seen in obese individuals.

Endotrophin is a peptide generated from ColVI in white adipose tissue. It has been shown to promote the growth of breast cancer cells and to induce insulin resistance. One study has demonstrated that baseline endotrophin levels offer predictive values indicating individuals who will show an optimized response to thiazolidinediones with respect to the lowering of HbA_{1c} and reduced risk of adverse side effects. Moreover, serum ETP level is independently associated with mortality in chronic kidney disease as well as being linked to chronic inflammation and fibrosis.

Given the many adverse effects of endotrophin on adipose tissue, inhibition of endotrophin may constitute an effective strategy to reduce fibrosis and enhance metabolic flexibility. In rodent models, a neutralizing antibody was shown to efficiently reduce endotrophin activity under high fat diet conditions, resulting in a significant improvement in insulin sensitivity (Sun et al., Nature Comm., 5:3485, 2014). Thus, improved methods of measuring endotrophin levels in samples from patients has significant utility in predicting or diagnosing disease, as well as potentially identifying subject for which pharmacologic intervention will be useful.

### SUMMARY

Thus, in accordance with the present disclosure, there is provided a method of detecting human endotrophin in a sample comprising:
(a) providing a surface coated with ETN4.1 (a.ka., ETH-4hu);
(b) contacting the coated surface with a sample;
(c) washing the coated surface;
(d) contacting the coated surface with a solution of ETN-1Rb;
(e) washing the coated surface;
(f) contacting the coated surface with a labeled antibody that binds selectively to ETN-1Rb;
(g) washing the coated surface; and
(h) determined labeled antibody binding to ETN-1Rb on the coated surface wherein a binding of the labeled antibody to the surface indicates the present of endotrophin in the sample. Washing steps (c), (e) and/or (g) may be repeated prior to the following step. The sample may be whole blood, plasma, serum, or tissue extract.

The method may further comprise treating the surface of step (a) with a buffered blocking solution comprising an inert protein prior to step (b), such as wherein the inert protein is albumin, e.g., bovine serum albumin, optionally at 3% wt/vol concentration. The solution of ETN-1Rb of step (d) may comprise an inert protein, e.g., albumin, such as bovine serum albumin, optionally at 3% wt/vol concentration.

The method may further comprise a step, prior to step (a), of coating the surface with a solution of ETN4.1 to produce the coated surface of step (a). The labeled antibody may be labeled with a ligand, an enzyme (e.g., horseradish peroxidase), a chromophore, a fluorophore, a dye, or a radiolabel.

The method may further comprise performing a positive control reaction and/or a negative control reaction and/or a standard curve reaction. Treating the surface of step (a) with a buffered blocking solution may be performed for about an hour. Coating the surface with a solution of ETN4.1 to produce the coated surface of step (a) may be performed for about 12-20 hours, such as about 16 hours. Step (f) may be performed for about 1-2 hours. Step (d) may be performed for about an hour.

Also provided is a kit comprising:
(a) antibody ETN4.1;
(b) antibody ETN-1Rb; and
(c) a labeled antibody that binds selectively to ETN-1Rb.

The ETN4.1 may be disposed on a surface, such as the surface of a well, a plate, or membrane. The kit may further comprise one or more buffered solutions or a reagent for making the same, one or more reagents for detection of the labeled antibody, and/or reagents for performing a positive and/or negative control reaction for binding of ENT4.1, ETN-1Rb and/or the labeled antibody.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Pictorial Representation of Assay Components and Steps.

FIGS. 2-7. Evaluation of Linear Nature of the Assay. Predictive relationship between OD₄₅₀ reading and endotrophin levels (ng/ml) in preparations containing recombinantly produced endotrophin protein in relationship plotted using the data collected in six Test sets (1-6), respectively, is show.

FIG. 8. Comparison of ETN Serum Concentrations between Breast Cancer Patients (mean±SEM: 35.58±0.96) and Healthy Subjects (mean±SEM: 12.98+1.56). Total of 6 assay replications, n=6.

FIGS. 9A-B. Evaluation of ETN4.5 Binding Specificity on Endotrophin: Immunohistochemistry (IHC) method. (FIG. 9A) Neutralization of ETN-4 (10 g/ml) binding to tissue endotrophin by pre-mixing ETN4.5 with 5 times (50 μg/ml) of endotrophin protein before staining detection. (FIG. 9B) ENT4.5 without pre-mixing with endotrophin protein.

FIG. 10. ETP and ENT4.1 Ab treatment of LX2 cells (Human Hepatic Stellate Cell Line).

ETP treatment was 1 μg/ml. Results show fibrotic response to endotrophin that can be neutralized with ENT4.1.

FIG. 11. Glucose uptake in Human Skeletal Myotube Cells. Endotrophin inhibits insulin-mediated glucose uptake and Ent 4.1 antibody neutralizes this effect. Apigenin is a generic glucose uptake inhibitor.

FIG. 12. Comparison of ENT4.1 and ENT4.5 Activity. Data shows that both ENT4.1 and ENT4.5 show comparable results for human serum endotrophin measurements and can be used interchangeably. Assays for 4.1 and 4.5 were performed on separate days on samples that were freeze/thawed in between, hence the slight variability in the absolute values.

### DESCRIPTION OF ILLUSTRATIVE EMBODIMENTS

As discussed above, endotrophin has been linked to a variety of biological conditions. As such, the inventors sought to develop a highly sensitive assay to detect and quantify endotrophin in samples from human subjects. They were able to create a highly sensitive (detection sensitivity at 0.4 ng/ml) and robust immunoassay. The assay accuracy is 95% with an average of coefficient of variation (CV) at 4.9% with standard deviation of 2.5% when comparing the actual values and calculated values using the assay. This is a novel quantitative assay for the measurement of endotrophin levels in human samples. These and other aspects of the disclosure are described in detail below.

### I. Endotrophin

Endotrophin is a cleavage product derived from the collagen VI(α3) chain. Collagen VI is expressed in a number of different tissues, but adipose tissue is a particularly prominent source for this extracellular matrix constituent. Mice lacking collagen VI are metabolically healthier due to reduced fibrosis in adipose tissue. Endotrophin appears to be a key player in collagen VI-mediated signaling effects, including its pro-fibrotic nature and chemoattractant properties for macrophages, while also playing an important role in cancer progression and the chemoresistance of tumor cells. It has been linked to inflammation, angiogenesis, fibrosis and epithelial-mesenchymal transition (EMT) in the context of cancer. It also seems to play a critical role in obesity-induced systemic insulin resistance by increasing chronic inflammation and fibrosis in adipose tissues.

Endotrophin is formed when collagen VI polypeptide chain α3 subunit releases its most C-terminal domain (C5) immediately after the secretion of microfilament tetramers to the extracellular surface (Sun et al., Diabetologia 60:24-29, 2017). Levels of endotrophin are upregulated in the adipose tissue of both diet-induced and genetically obese mouse models. Obese patients with insulin resistance display higher levels of endotrophin than obese individuals with normal insulin sensitivity, suggesting that endotrophin is a useful biomarker for the level of metabolic fitness during the development of obesity.

The significance of an excess of local endotrophin overexpression have been studied in different animal models (Sun et al., Nature Comm., 5:3485, 2014). The findings indicate that endotrophin is associated with an unfavorable microenvironment in obese adipose tissue, and also a driving force development of systemic insulin resistance and other metabolic disorders (Sun et al., Nature Comm., 5:3485, 2014). Specifically, overexpression of endotrophin in obese adipose tissue stimulates fibrosis by upregulating ECM constituents (Sun et al., Nature Comm., 5:3485, 2014). Moreover, through potent chemoattractant activity, it leads to macrophage accumulation and an enhanced proinflammatory microenvironment in adipose tissue. The pro-fibrotic and proinflammatory effects of endotrophin can contribute to metabolic dysfunction in adipose tissue that in turn leads to systemic insulin resistance (Sun et al., J. Clin. Invest. 121:2094, 2011; Sun et al., Cell Metabol. 18:470, 2013).

One contributory mechanism for endotrophin may involve enhanced macrophage accumulation in endotrophin-enriched adipose tissue. Macrophages are postulated to contribute to the development of insulin resistance (Sun et al., J. Clin. Invest. 121:2094). In addition, the local fibrosis induced by endotrophin causes significant mechanical stress, limiting the ability of adipose tissue to take up and esterify NEFAs, eventually leading to hepatic steatosis (Sun et al., Cell Metabol. 18:470, 2013). Endotrophin-induced fibrosis has also been shown to promote local dysfunction in brown adipose tissue (Sun et al., Nature Comm., 5:3485, 2014).

### II. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgAQ1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md., 1991); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917, 1987); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212, 1999; Ruiz, M. et al. Nucl. Acids Res. 28:219-221, 2000). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670, 2001).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

It will be understood that monoclonal antibodies binding to endotrophin will have several applications. These include the production of diagnostic kits for use in detecting and diagnosing endotrophin related disease, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as ASO1 or ASO3). Additional experimental forms of inoculation to induce endotrophin-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259: Engen and Smith (2001) Anal. Chem. 73: 256A-265A.

The term “epitope” refers to a site on an antigen to which B and/or 1 cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-endotrophin antibody, the above-described binding methodology is performed in two orientations: In a first orientation, the reference antibody is allowed to bind to endotrophin under saturating conditions followed by assessment of binding of the test antibody to endotrophin. In a second orientation, the test antibody is allowed to bind to endotrophin molecule under saturating conditions followed by assessment of binding of the reference antibody to endotrophin. If, in both orientations, only the first (saturating) antibody is capable of binding to endotrophin, then it is concluded that the test antibody and the reference antibody compete for binding to endotrophin. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains, or complete light and heavy chains, as show below.

TABLE A
CDR Sequences of Antibody Light Chains

| Antibody Name | | CDR1-LC | CDR2-LC | CDR3-LC |
|---|---|---|---|---|
| ETN- | DNA | cagagca | gatgc | caacaggg |
| Mab- | | ttggtag | atcg | ttatagtg |
| 1Rb | | taat | | ataattat |
| | | (SEQ ID | | cttgataa |
| | | NO: 1) | | tgct |
| | | | | (SEQ ID |
| | | | | NO: 2) |
| | AA | QSIGSN | DAS | QQGYSDN |
| | | (SEQ ID | | YLDNA |
| | | NO: 3) | | (SEQ ID |
| | | | | NO: 4) |
| ETN4.1 | DNA | cagagta | aaggc | caatatag |
| | | ttagtag | atcc | tgattggg |
| | | tagctac | | ctaatagt |
| | | (SEQ ID | | tatgggaa |
| | | NO: 5) | | tgct |
| | | | | (SEQ ID |
| | | | | NO: 6) |
| | AA | QSISSSY | KAS | QYSDWA |
| | | (SEQ ID | | NSYGNA |
| | | NO: 7) | | (SEQ ID |
| | | | | NO: 8) |

TABLE B
CDR Sequences of Antibody Heavy Chains

| Antibody Name | | CDR1-LC | CDR2-LC | CDR3-LC |
|---|---|---|---|---|
| ETN- | DNA | ggaatcg | atttatgc | gcgagaga |
| Mab- | | acttcag | tggtcgta | tggggcta |
| 1Rb | | tgccgg | gtcttaac | gcagtggc |
| | | ctacta | act | tactactt |
| | | c | (SEQ ID | taagttg |
| | | (SEQ ID | NO: 10) | (SEQ ID |
| | | NO: 9) | | NO: 11) |
| | AA | GIDFSA | IYAGRS | ARDGASS |
| | | GYY | LNT | GYYFKL |
| | | (SEQ ID | (SEQ ID | (SEQ ID |
| | | NO: 12) | NO: 13) | NO: 14) |
| ETN4.1 | DNA | ggattct | atttatgg | agaaaag |
| | | ccttcag | tggtaata | atattaa |
| | | tagcggct | ataaccca | tattggt |
| | | actac | (SEQ ID | ggtgctt |
| | | (SEQ ID | NO: 16) | atgagttg |
| | | NO: 15) | | (SEQ ID |
| | | | | NO: 17) |
| | AA | GFSFSS | IYGGNNNP | RKDINI |
| | | GYY | (SEQ ID | GGAYEL |
| | | (SEQ ID | NO: 19) | (SEQ ID |
| | | NO: 18) | | NO: 20) |

TABLE C
Heavy and Light Chain Variable Sequences

| | | |
|---|---|---|
| ETN4.1 | Heavy | GAGGTCCAGCTGCTGGAGAGCGGAGGAGGACTGGTGCAGCCCGGA |
| | | GGATCTTTAAGACTGAGCTGTGCCGCCAGCGGCTTCAGCTTCAGC |
| | | AGCGGCTACTACATCTGCTGGGTGAGACAAGCTCCCGGTAAAGGT |
| | | TTAGAGTGGATCGCTTGTATCTACGGCGGCAACAACAACCCCTAC |
| | | TACGCCAACTGGGTGAACGGCAGATTCACCATCTCTCGTGACAAC |
| | | AGCAAGAACACTTTATATTTACAGATGAACTCTTTAAGGGCCGAG |
| | | GACACCGCCGTGTACTACTGCGCTCGTAAGGACATCAACATCGGC |
| | | GGCGCCTATGAGCTGTGGGGCCAAGGTACTTTAGTGACCGTGAGC |
| | | AGC (SEQ ID NO: 21) |
| | Light | GACATCCAGATGACCCAGAGCCCTAGCTCITTAAGCGCCAGCGTG |
| | | GGCGACAGAGTGACCATCACTTGTCAAGCTAGCCAGAGCATCAGC |
| | | AGCAGCTATTTAAGCTGGTACCAGCAGAAGCCCGGCAAGGCCCCC |
| | | AAGCTGCTGATCTACAAGGCCAGCACACTGGCCAGCGGCGTGCCT |
| | | TCTCGTTTTAGCGGCAGCGGCAGCGGAACCGACTTCACTTTAACC |
| | | ATCAGCTCTTTACAGCCCGAGGACTTCGCCACCTACTACTGCCAG |
| | | TACAGCGACTGGGCCAACAGCTATGGCAACGCCTTCGGCGGCGGC |
| | | ACCAAGGTGGAGATCAAG (SEQ ID NO: 22) |
| ETN4.1 | Heavy | EVQLLESGGGLVQPGGSLRLSCAASGFSFSSGYYICWVRQAPGKG |
| | | LEWIACIYGGNNNPYYANWVNGRFTISRDNSKNTLYLQMNSLRAE |
| | | DTAVYYCARKDINIGGAYELWGQGTLVTVSS (SEQ ID NO: 23) |
| | Light | DIQMTQSPSSLSASVGDRVTITCQASQSISSSYLSWYQQKPGKAP |
| | | KLLIYKASTLASGVPSRFSGSGSGTDFTLTISSLQPEDFATYYCQ |
| | | YSDWANSYGNAFGGGTKVEIK (SEQ ID NO: 24) |
| ETN- | Heavy | CAGTCGGTGAAGGAGTCCGGGGGAGACCTGGTCAAGCCTGGGG |
| 1Rb | | CTTCCCTGACACTCACCTGCACAGCCTCTGGAATCGACTTCAG |
| | | TGCCGGCTACTACATGTGCTGGGTCCGCCAGGCTCCAGGGAAG |
| | | GGGCTGGAGTTGATCGCATGCATTTATGCTGGTCGTAGTCTTA |
| | | ACACTTTCTACGCGAGCTGGGCGAAAGGCCGATTCACCATCTC |
| | | CAGAGCCTCGTCGACCACGGTGACTCTGGCGATGACCAGTCTG |
| | | ACAGCCGCGGACACGGCCACCTATTTCTGTGCGAGAGATGGGG |
| | | CTAGCAGTGGCTACTACTTTAAGTIGTGGGGCCCAGGCACCCT |
| | | GGTCACCATCTCTTCA (SEQ ID NO: 25) |
| | Light | GAGCTCGATATGACCCAGACTCCAGCCTCTGTGGAGGTAGCTG |
| | | TGGGAGGCACAGTCACCATCAAGTGCCAGGCCAGTCAGAGCAT |
| | | TGGTAGTAATTTAGCCTGGTATCAGCAGAAACCAGGGCAGCGT |
| | | CCCAATGTCCTGATCTACGATGCATCGAATCTGGCATCTGGGG |
| | | TCTCATCGCGGTTCAAAGGCAGTGGATCTGGGACACAGTTCAC |
| | | TCTCACCATCAGCGGCGGGGAGTGTGCCGATGCTGCCACTTAC |
| | | TACTGTCAACAGGGTTATAGTGATAATTATCTTGATAATGCTT |
| | | TCGGCGGAGGGACCGAGGTGGTGGTCAAA (SEQ ID NO: 26) |
| ETN- | Heavy | QSVKESGGDLVKPGASLTLTCTASGIDFSAGYYMCWVRQAPGK |
| 1Rb | | GLELIACIYAGRSLNTFYASWAKGRFTISRASSTTVTLAMTSL |
| | | TAADTATYFCARDGASSGYYFKLWGPGTLVTISS (SEQ ID |
| | | NO: 27) |
| | Light | ELDMTQTPASVEVAVGGTVTIKCQASQSIGSNLAWYQQKPGQR |
| | | PNVLIYDASNLASGVSSRFKGSGSGTQFTLTISGGECADAATY |
| | | YCQQGYSDNYLDNAFGGGTEVVVK (SEQ ID NO: 28) |

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge, E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### C. Purification

In certain embodiments, the antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Immunodetection Methods

In still further embodiments, the present disclosure concerns immunodetection methods for binding, purifying, removing, quantifying and otherwise generally detecting endotrophin and its associated antigens. A wide variety of assay formats are contemplated, but specifically those that would be used to detect endotrophin in a fluid obtained from a subject, such as saliva, blood, plasma, sputum, semen or urine. The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing endotrophin, and contacting the sample with a first antibody in accordance with the present disclosure, as the case may be, under conditions effective to allow the formation of immunocomplexes.

These methods include methods for purifying endotrophin from a sample. The antibody will preferably be linked to a solid support, such as in the form of a column matrix, and the sample suspected of containing endotrophin will be applied to the immobilized antibody. The unwanted components will be washed from the column, leaving the endotrophin immunocomplexed to the immobilized antibody, which is then collected by removing the endotrophin from the column.

The immunobinding methods also include methods for detecting and quantifying the amount of endotrophin in a sample and the detection and quantification of any immune complexes formed during the binding process. Here, one would obtain a sample suspected of containing endotrophin and contact the sample with an antibody that binds endotrophin, followed by detecting and quantifying the amount of immune complexes formed under the specific conditions. In terms of antigen detection, the biological sample analyzed may be any sample that is suspected of containing endotrophin, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to endotrophin present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme-linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing endotrophin is added to the wells. After binding and washing to remove non-specifically bound immune complexes, the bound endotrophin may be detected.

Detection may be achieved by the addition of another anti-endotrophin antibody that is linked to a detectable label. This type of ELISA is a simple “sandwich ELISA.” Detection may also be achieved by the addition of a second anti-endotrophin antibody, followed by the addition of a third antibody that has binding affinity for the second antibody, with the third antibody being linked to a detectable label.

In another exemplary ELISA, the samples suspected of containing endotrophin are immobilized onto the well surface and then contacted with the anti-endotrophin antibodies of the disclosure. After binding and washing to remove non-specifically bound immune complexes, the bound anti-endotrophin antibodies are detected. Where the initial anti-endotrophin antibodies are linked to a detectable label, the immune complexes may be detected directly. Again, the immune complexes may be detected using a second antibody that has binding affinity for the first anti-endotrophin antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzothiazoline-6-sulfonic acid (ABTS), or H₂O₂, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

### D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### E. Immunodetection Kits

In still further embodiments, the present disclosure concerns immunodetection kits for use with the immunodetection methods described above. As the antibodies may be used to detect endotrophin, the antibodies may be included in the kit. The immunodetection kits will thus comprise, in suitable container means, a first antibody that binds to endotrophin, and optionally an immunodetection reagent.

In certain embodiments, the anti-endotrophin antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for endotrophin, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of endotrophin, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### IV. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Results

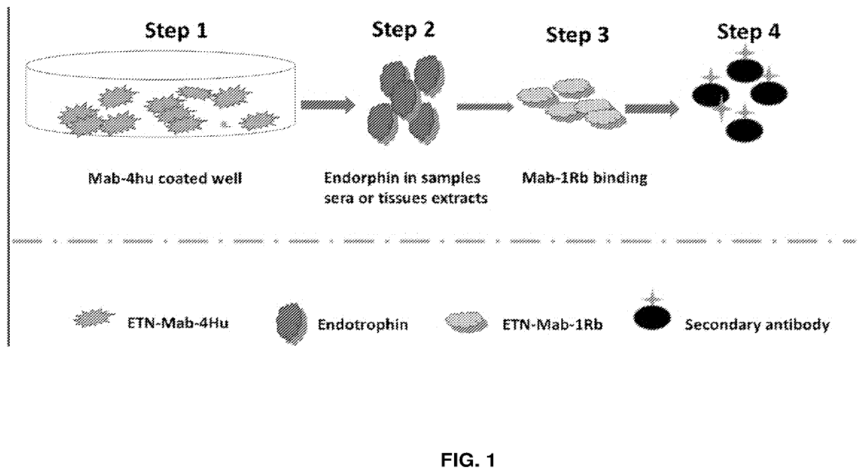

Assay design. FIG. 1 shows an immunoassay using two monoclonal antibodies specifically recognizing endotrophin.

Description of assay procedures. Step 1: High binding plates (96-well or 384 wells) are first coated with ETN4.1 at 2 μg/ml in phosphate-buffered saline (PBS, pH 7.4), 100 pI/well, at 4° C. for >16 hours (overnight), with plate covered. Step 2: Remove coating solution, and add 3% of BSA in PBS, 200 μL/well for 1 hour at 25° C. (room temperature). Step 3: Remove blocking solution and wash 3 times with PBS-T (0.05% Tween 20 in PBS), and 2 times with PBS, 200 pI/per well. Washing step can be done manually or using a plate washer. Step 4: Add 100 pI/well 1:20 to 1:200 diluted sera or tissue extracts (depending concentrations of samples) into each well and incubate for 1 hours at room temperature. For standard curve development, add 100 μl/per well of series of 2-fold titrated endotrophin recombinant protein (Sino Biologicals, Col6A3, catalog #16125-H07H) starting concentration at 50 ng/ml (in PBS with 1% BSA) and incubating for 1 hour at room temperature. Step 5: Repeat washing step (Step 4), then add 100 μl/well of 1 g/mL of ETN-1Rb (in PBS with 1% BSA) into each well and incubate for 1.5 hours at room temperature. Step 6: After washing as step 4, add 100 μl/well 1:5000 diluted anti-Rabbit Fab2-HRP antibody (Jackson ImmunoResearch) into each well, incubate for 1 hour at room temperature. Step 7: Repeat washing step 4, then add 100 μl/well of TMB substrate solution (Thermo Fisher) into each well, incubation for 8 minutes, then add 50 μl/well of 2M of H₂SO₄ to stop reaction before reading plates. Step 8: Measure absorbance at 450 nm using a 96-well plate reader and develop standard curve using data from endotrophin recombinant protein to calculate endotrophin in testing samples.

Validation of ETN4.5 specificity on endotrophin using immune histochemistry (IHC) method. Validation is shown in FIGS. 9A-B.

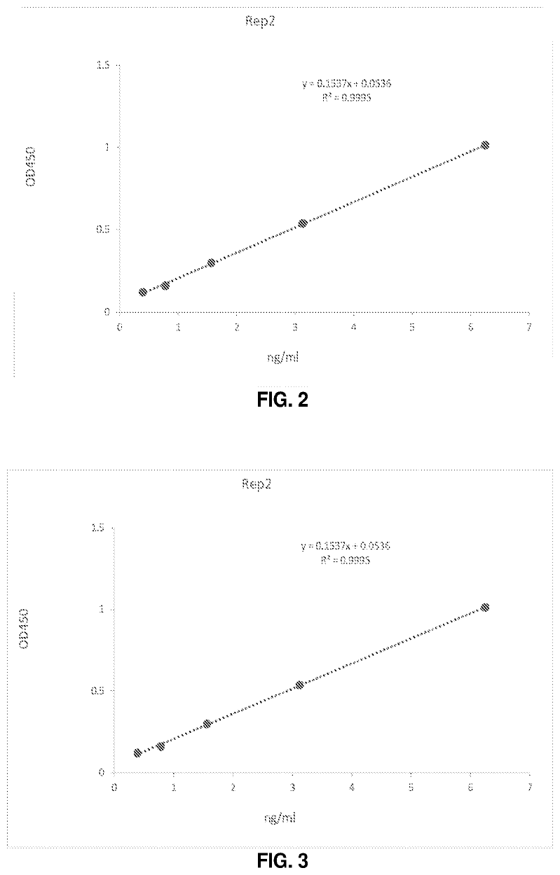

Evaluation of assay precision and coefficient of variations (CV) using recombinantly produced endotrophin protein. The following data relate to FIGS. 2-7.

TABLE 1
data of test set 1 (plot in FIG. 2)

| Actual | OD450 nM | Calculated | | | |
|---|---|---|---|---|---|
| value | (Reading | value | Mean | STD | CV |
| (ng/ml) | signal) | (ng/ml) | (ng/ml) | (ng/ml) | (%) |
| 6.25 | 1.317 | 6.22 | 6.23 | 0.02 | 0.35 |
| 3.13 | 0.705 | 3.22 | 3.17 | 0.06 | 2.03 |
| 1.56 | 0.360 | 1.53 | 1.54 | 0.03 | 1.69 |
| 0.78 | 0.200 | 0.74 | 0.76 | 0.03 | 4.03 |
| 0.39 | 0.133 | 0.41 | 0.40 | 0.02 | 3.80 |
| | | | | Average | 2.38 |
| | | | | CV (%) | |

TABLE 2
Data of test set 2 (see FIG. 3)

| Actual | | Calculated | | | |
|---|---|---|---|---|---|
| value | | value | Mean | STD | CV |
| (ng/ml) | Rep2-OD | (ng/ml) | (ng/ml) | (ng/ml) | (%) |
| 6.25 | 1.013 | 6.24 | 6.24 | 0.01 | 0.13 |
| 3.13 | 0.537 | 3.15 | 3.14 | 0.02 | 0.48 |
| 1.56 | 0.299 | 1.60 | 1.58 | 0.02 | 1.47 |
| 0.78 | 0.160 | 0.69 | 0.74 | 0.06 | 8.41 |
| 0.39 | 0.120 | 0.43 | 0.41 | 0.03 | 7.43 |
| | | | | Average | 3.58 |
| | | | | CV (%) | |

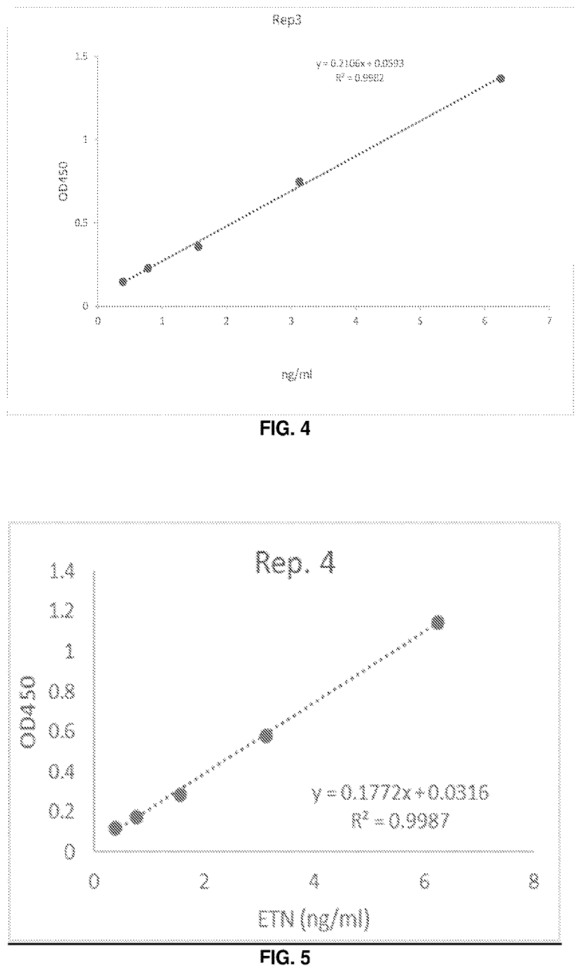

Data of test set 3 (see FIG. 4)

| Actual | | Calculated | | | |
|---|---|---|---|---|---|
| value | | value | Mean | STD | CV |
| (ng/ml) | Rep3-OD | (ng/ml) | (ng/ml) | (ng/ml) | (%) |
| 6.25 | 1.368 | 6.21 | 6.23 | 0.03 | 0.41 |
| 3.13 | 0.746 | 3.26 | 3.19 | 0.10 | 2.99 |
| 1.56 | 0.358 | 1.42 | 1.49 | 0.10 | 6.91 |
| 0.78 | 0.229 | 0.81 | 0.79 | 0.02 | 2.12 |
| 0.39 | 0.146 | 0.41 | 0.40 | 0.02 | 3.87 |
| | | | | Average | 3.26 |
| | | | | CV (%) | |

Data of test set 4 (see FIG. 5)

| Actual | | Calculated | | | |
|---|---|---|---|---|---|
| value | | value | Mean | STD | CV |
| (ng/ml) | Rep4-OD | (ng/ml) | (ng/ml) | (ng/ml) | (%) |
| 6.25 | 1.146 | 6.29 | 6.27 | 0.03 | 0.43 |
| 3.13 | 0.579 | 3.09 | 3.11 | 0.02 | 0.80 |
| 1.56 | 0.286 | 1.44 | 1.50 | 0.09 | 5.90 |
| 0.78 | 0.173 | 0.80 | 0.79 | 0.01 | 1.50 |
| 0.39 | 0.119 | 0.49 | 0.44 | 0.07 | 16.26 |

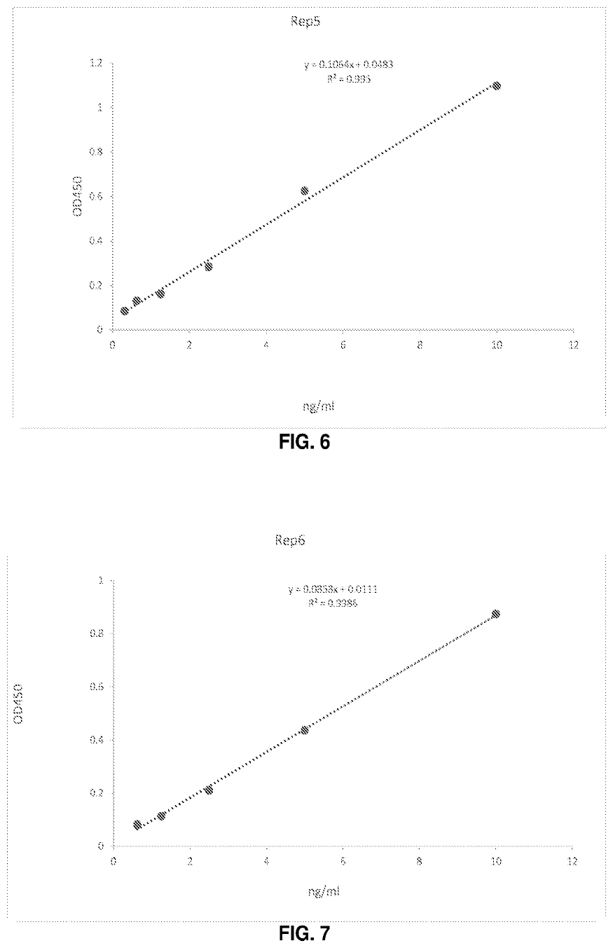

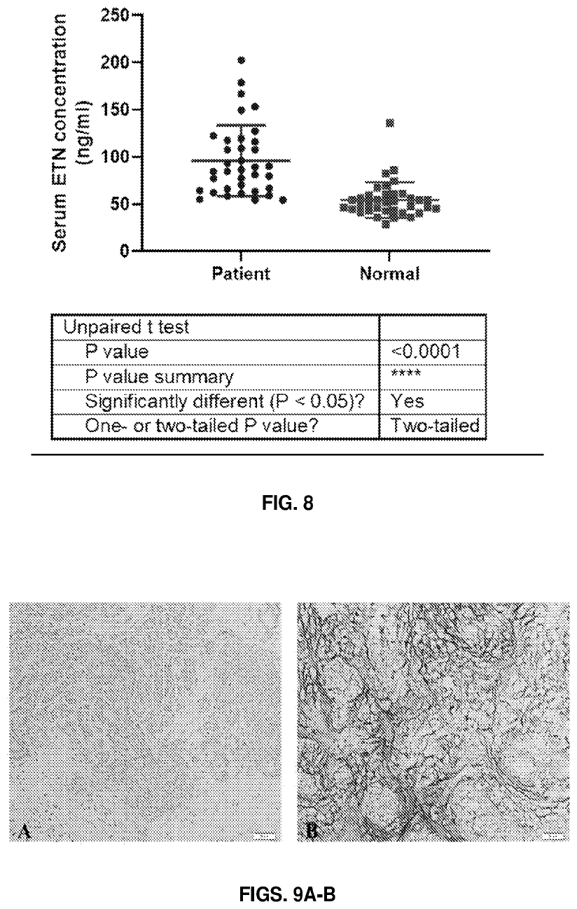

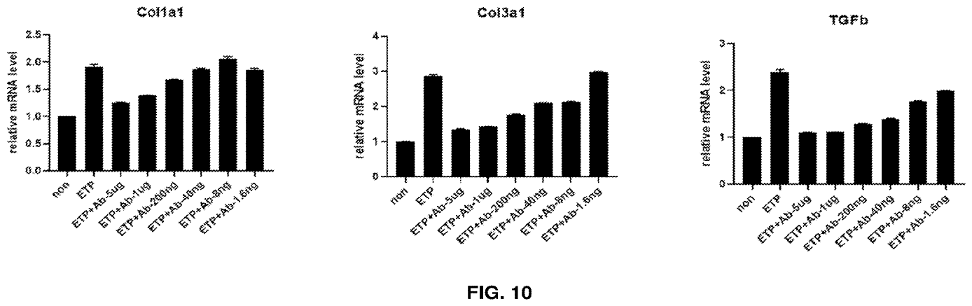

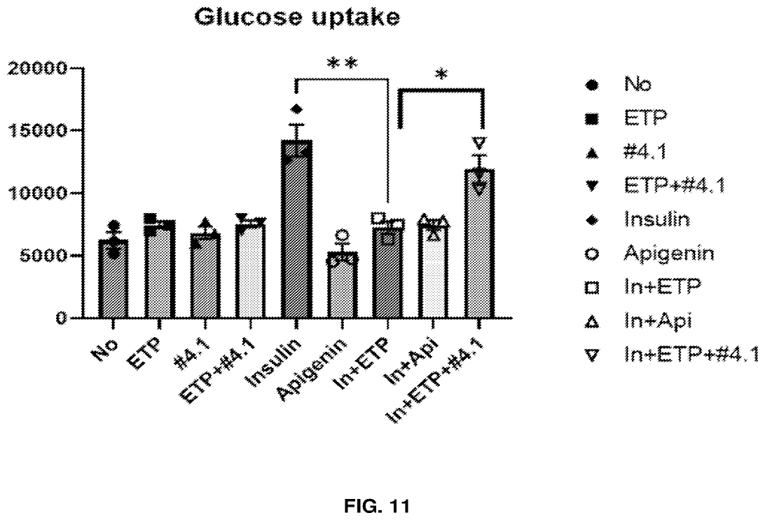

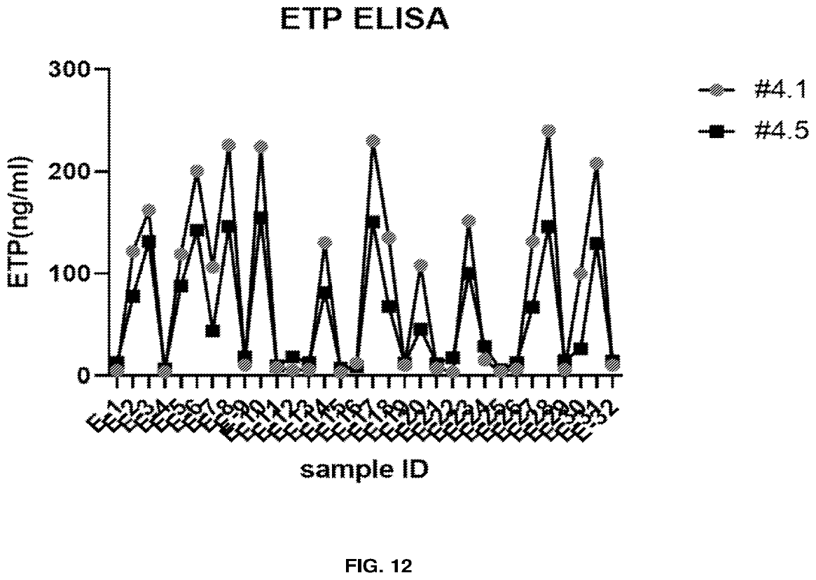

Data of test set 5 (see FIG. 6)

| Actual | | Calculated | | | |
|---|---|---|---|---|---|
| value | | value | Mean | STD | CV |
| (ng/ml) | Rep6-OD | (ng/ml) | (ng/ml) | (ng/ml) | (%) |
| 10.00 | 0.874 | 10.06 | 10.03 | 0.04 | 0.40 |
| 5.00 | 0.437 | 4.96 | 4.98 | 0.03 | 0.53 |
| 2.50 | 0.211 | 2.33 | 2.41 | 0.12 | 5.02 |
| 1.25 | 0.114 | 1.19 | 1.22 | 0.04 | 3.20 |
| 0.63 | 0.083 | 0.83 | 0.73 | 0.15 | 20.30 |
| | | | | Average | 5.89 |
| | | | | CV (%) | |

Data of test set 6 (see FIG. 7)

| Actual | | Calculated | | | |
|---|---|---|---|---|---|
| value | | value | Mean | STD | CV |
| (ng/ml) | Rep6-OD | (ng/ml) | (ng/ml) | (ng/ml) | (%) |
| 10.00 | 0.874 | 10.06 | 10.03 | 0.04 | 0.40 |
| 5.00 | 0.437 | 4.96 | 4.98 | 0.03 | 0.53 |
| 2.50 | 0.211 | 2.33 | 2.41 | 0.12 | 5.02 |
| 1.25 | 0.114 | 1.19 | 1.22 | 0.04 | 3.20 |
| 0.63 | 0.083 | 0.83 | 0.73 | 0.15 | 20.30 |
| | | | | Average | 4.98 |
| | | | | CV (%) | |

TABLE 3
Summary of assay variations (n = 6 test sets; n = 30 data points)

| | Average |
|---|---|
| Test set | CV (%) |
| 1 | 2.381 |
| 2 | 3.584 |
| 3 | 3.263 |
| 4 | 4.977 |
| 5 | 9.359 |
| 6 | 5.890 |
| Mean of test | 4.909 |
| set CV (%) | |
| SD | 2.515 |

Assay set using serum samples from breast cancer patients in comparison with serum from healthy people. Data are shown in the following tables.

TABLE 4
Serum endotrophin concentrations determined using standard curve derived from test set (1 & 2)

| Set 1 | Rep 1-OD | | Serum | Rep 2-OD | | Serum | Mean | | |
|---|---|---|---|---|---|---|---|---|---|
| Sample | (dilution | Calculated | level | (dilution | Calculated | level | (rep1, 2) | | CV |
| Name | 50X) | (ng/ml) | (ng/ml) | 100X) | (ng/ml) | (ng/ml) | ng/ml | STD | (%) |
| P1 | 0.3247 | 1.35 | 67.6 | 0.2926 | 1.55 | 155.5 | 111.5 | 62.18 | 55.75 |
| P2 | 0.5923 | 2.66 | 133.2 | 0.3175 | 1.72 | 171.7 | 152.5 | 27.21 | 17.85 |
| P3 | 0.521 | 2.31 | 115.7 | 0.3074 | 1.65 | 165.1 | 140.4 | 34.93 | 24.88 |
| P4 | 1.1313 | 5.31 | 265.5 | 0.7966 | 4.83 | 483.4 | 374.4 | 154.12 | 41.16 |
| P5 | 0.3774 | 1.61 | 80.5 | 0.2441 | 1.24 | 123.9 | 102.2 | 30.72 | 30.05 |
| P6 | 0.7906 | 3.64 | 181.9 | 0.4344 | 2.48 | 247.8 | 214.8 | 46.59 | 21.69 |
| P7 | 0.5099 | 2.26 | 113.0 | 0.3686 | 2.05 | 204.9 | 159.0 | 65.01 | 40.90 |
| P8 | 0.801 | 3.69 | 184.4 | 0.486 | 2.81 | 281.3 | 232.9 | 68.52 | 29.42 |
| P9 | 0.7201 | 3.29 | 164.6 | 0.3977 | 2.24 | 223.9 | 194.2 | 41.93 | 21.59 |
| P10 | 0.6565 | 2.98 | 149.0 | 0.5198 | 3.03 | 303.3 | 226.1 | 109.14 | 48.26 |
| P11 | 0.663 | 3.01 | 150.6 | 0.553 | 3.25 | 324.9 | 237.7 | 123.29 | 51.86 |
| P12 | 0.3614 | 1.53 | 76.6 | 0.3167 | 1.71 | 171.2 | 123.9 | 66.90 | 54.00 |
| P13 | 0.454 | 1.99 | 99.3 | 0.3302 | 1.80 | 180.0 | 139.6 | 57.04 | 40.86 |
| P14 | 0.3598 | 1.52 | 76.2 | 0.3026 | 1.62 | 162.0 | 119.1 | 60.69 | 50.96 |
| P15 | 0.4885 | 2.16 | 107.8 | 0.3461 | 1.90 | 190.3 | 149.0 | 58.37 | 39.17 |
| P16 | 0.3656 | 1.55 | 77.6 | 0.2153 | 1.05 | 105.2 | 91.4 | 19.52 | 21.36 |
| P17 | 0.4479 | 1.96 | 97.8 | 0.3029 | 1.62 | 162.2 | 130.0 | 45.54 | 35.03 |
| P18 | 1.0303 | 4.81 | 240.7 | 0.4872 | 2.82 | 282.1 | 261.4 | 29.30 | 11.21 |
| P19 | 0.964 | 4.49 | 224.4 | 0.6196 | 3.68 | 368.2 | 296.3 | 101.71 | 34.32 |
| P20 | 0.4004 | 1.72 | 86.1 | 0.2388 | 1.20 | 120.5 | 103.3 | 24.29 | 23.51 |
| P21 | 0.6688 | 3.04 | 152.0 | 0.4377 | 2.50 | 249.9 | 200.9 | 69.24 | 34.46 |
| P22 | 0.5773 | 2.59 | 129.5 | 0.3986 | 2.24 | 224.5 | 177.0 | 67.12 | 37.92 |
| P23 | 0.7056 | 3.22 | 161.0 | 0.4718 | 2.72 | 272.1 | 216.6 | 78.54 | 36.27 |
| P24 | 0.4018 | 1.73 | 86.5 | 0.271 | 1.41 | 141.4 | 114.0 | 38.86 | 34.10 |
| P25 | 0.6321 | 2.86 | 143.0 | 0.2929 | 1.56 | 155.7 | 149.3 | 8.99 | 6.02 |
| P26 | 0.3539 | 1.49 | 74.7 | 0.1991 | 0.95 | 94.7 | 84.7 | 14.10 | 16.64 |
| P27 | 0.3521 | 1.49 | 74.3 | 0.2177 | 1.07 | 106.8 | 90.5 | 22.97 | 25.37 |
| P28 | 0.7196 | 3.29 | 164.5 | 0.5208 | 3.04 | 304.0 | 234.2 | 98.65 | 42.12 |
| P29 | 0.3324 | 1.39 | 69.5 | 0.1953 | 0.92 | 92.2 | 80.8 | 16.08 | 19.89 |
| P30 | 1.0574 | 4.95 | 247.3 | 0.6002 | 3.56 | 355.6 | 301.5 | 76.58 | 25.40 |
| P31 | 0.5916 | 2.66 | 133.0 | 0.2744 | 1.44 | 143.7 | 138.4 | 7.50 | 5.42 |
| P32 | 0.526 | 2.34 | 117.0 | 0.2808 | 1.48 | 147.8 | 132.4 | 21.83 | 16.49 |
| P33 | 0.2807 | 1.14 | 56.8 | 0.2052 | 0.99 | 98.6 | 77.7 | 29.60 | 38.10 |
| P34 | 0.3316 | 1.39 | 69.3 | 0.2731 | 1.43 | 142.8 | 106.0 | 52.01 | 49.05 |
| P35 | 0.2359 | 0.92 | 45.8 | 0.2653 | 1.38 | 137.7 | 91.8 | 65.02 | 70.86 |
| P36 | 0.318 | 1.32 | 65.9 | 0.2557 | 1.31 | 131.5 | 98.7 | 46.36 | 46.97 |
| N1 | 0.2187 | 0.83 | 41.6 | 0.1439 | 0.59 | 58.8 | 50.2 | 12.16 | 24.24 |
| N2 | 0.2733 | 1.10 | 55.0 | 0.1676 | 0.74 | 74.2 | 64.6 | 13.59 | 21.04 |
| N3 | 0.1822 | 0.65 | 32.6 | 0.1575 | 0.68 | 67.6 | 50.1 | 24.74 | 49.39 |
| N4 | 0.2223 | 0.85 | 42.4 | 0.1699 | 0.76 | 75.7 | 59.1 | 23.49 | 39.78 |
| N5 | 0.1121 | 0.31 | 15.4 | 0.1183 | 0.42 | 42.1 | 28.8 | 18.87 | 65.64 |
| N6 | 0.1506 | 0.50 | 24.9 | 0.1735 | 0.78 | 78.0 | 51.4 | 37.59 | 73.08 |
| N7 | 0.1476 | 0.48 | 24.1 | 0.1351 | 0.53 | 53.0 | 38.6 | 20.44 | 53.00 |
| N8 | 0.3558 | 1.50 | 75.2 | 0.1957 | 0.92 | 92.5 | 83.8 | 12.20 | 14.56 |
| N9 | 0.201 | 0.74 | 37.2 | 0.1495 | 0.62 | 62.4 | 49.8 | 17.80 | 35.74 |
| N10 | 0.205 | 0.76 | 38.2 | 0.1385 | 0.55 | 55.2 | 46.7 | 12.05 | 25.79 |
| N11 | 0.3101 | 1.28 | 64.0 | 0.1981 | 0.94 | 94.0 | 79.0 | 21.23 | 26.88 |
| N12 | 0.2877 | 1.17 | 58.5 | 0.182 | 0.84 | 83.5 | 71.0 | 17.71 | 24.94 |
| N13 | 0.2307 | 0.89 | 44.5 | 0.2496 | 1.28 | 127.5 | 86.0 | 58.70 | 68.25 |
| N14 | 0.2606 | 1.04 | 51.8 | 0.2297 | 1.15 | 114.6 | 83.2 | 44.36 | 53.31 |
| N15 | 0.2307 | 0.89 | 44.5 | 0.286 | 1.51 | 151.2 | 97.9 | 75.45 | 77.10 |
| N16 | 0.231 | 0.89 | 44.6 | 0.2041 | 0.98 | 97.9 | 71.2 | 37.72 | 52.94 |
| N17 | 0.3271 | 1.36 | 68.2 | 0.2676 | 1.39 | 139.2 | 103.7 | 50.26 | 48.47 |
| N18 | 0.2293 | 0.88 | 44.2 | 0.57 | 3.36 | 336.0 | 190.1 | 206.35 | 108.56 |
| N19 | 0.2783 | 1.12 | 56.2 | 0.5056 | 2.94 | 294.1 | 175.1 | 168.22 | 96.05 |
| N20 | 0.226 | 0.87 | 43.4 | 0.261 | 1.35 | 134.9 | 89.1 | 64.76 | 72.65 |
| N21 | 0.2165 | 0.82 | 41.0 | 0.3396 | 1.86 | 186.1 | 113.5 | 102.57 | 90.33 |
| N22 | 0.2796 | 1.13 | 56.5 | 0.3495 | 1.93 | 192.5 | 124.5 | 96.18 | 77.25 |
| N23 | 0.1076 | 0.29 | 14.3 | 0.3956 | 2.23 | 222.5 | 118.4 | 147.23 | 124.34 |
| N24 | 0.2899 | 1.18 | 59.0 | 0.2442 | 1.24 | 124.0 | 91.5 | 45.95 | 50.21 |
| N25 | 0.1954 | 0.72 | 35.8 | 0.149 | 0.62 | 62.1 | 49.0 | 18.54 | 37.88 |
| N26 | 0.7785 | 3.58 | 178.9 | 0.4819 | 2.79 | 278.7 | 228.8 | 70.54 | 30.83 |
| N27 | 0.2659 | 1.06 | 53.1 | 0.1731 | 0.78 | 77.7 | 65.4 | 17.40 | 26.59 |
| N28 | 0.1973 | 0.73 | 36.3 | 0.1777 | 0.81 | 80.7 | 58.5 | 31.42 | 53.68 |
| N29 | 0.2359 | 0.92 | 45.8 | 0.1736 | 0.78 | 78.1 | 61.9 | 22.84 | 36.87 |
| N30 | 0.2181 | 0.83 | 41.4 | 0.1534 | 0.65 | 64.9 | 53.2 | 16.63 | 31.28 |
| N31 | 0.2531 | 1.00 | 50.0 | 0.2094 | 1.01 | 101.4 | 75.7 | 36.32 | 47.99 |
| N32 | 0.2407 | 0.94 | 47.0 | 0.1866 | 0.87 | 86.5 | 66.7 | 27.98 | 41.93 |
| N33 | 0.2428 | 0.95 | 47.5 | 0.1604 | 0.69 | 69.5 | 58.5 | 15.57 | 26.62 |
| N34 | 0.202 | 0.75 | 37.5 | 0.1663 | 0.73 | 73.3 | 55.4 | 25.36 | 45.78 |
| N35 | 0.1749 | 0.62 | 30.8 | 0.1398 | 0.56 | 56.1 | 43.4 | 17.87 | 41.12 |
| N36 | 0.1585 | 0.54 | 26.8 | 0.139 | 0.56 | 55.6 | 41.2 | 20.34 | 49.41 |

TABLE 5
Serum ENT concentrations determined using standard curve derived from test set (3 & 4)

| Set 2 | Rep 3-OD | | Serum | Rep 4-OD | | Serum | | | |
|---|---|---|---|---|---|---|---|---|---|
| Sample | (dilution | Calculated | level | (dilution | Calculated | level | Mean | STD | CV |
| Name | 50X) | (ng/ml) | (ng/ml) | 100X) | (ng/ml) | (ng/ml) | (ng/ml) | (ng/ml) | (%) |
| P1 | 0.2869 | 1.081 | 54.0 | 0.1668 | 0.763 | 76.3 | 65.2 | 15.7 | 24.2 |
| P2 | 0.2364 | 0.841 | 42.0 | 0.1577 | 0.712 | 71.2 | 56.6 | 20.6 | 36.4 |
| P3 | 0.2447 | 0.880 | 44.0 | 0.1357 | 0.587 | 58.7 | 51.4 | 10.4 | 20.3 |
| P4 | 0.4893 | 2.042 | 102.1 | 0.2389 | 1.170 | 117.0 | 109.5 | 10.5 | 9.6 |
| P5 | 0.2134 | 0.732 | 36.6 | 0.1187 | 0.492 | 49.2 | 42.9 | 8.9 | 20.7 |
| P6 | 0.3894 | 1.567 | 78.4 | 0.1794 | 0.834 | 83.4 | 80.9 | 3.6 | 4.4 |
| P7 | 0.4056 | 1.644 | 82.2 | 0.1845 | 0.863 | 86.3 | 84.3 | 2.9 | 3.4 |
| P8 | 0.3197 | 1.236 | 61.8 | 0.1749 | 0.809 | 80.9 | 71.3 | 13.5 | 18.9 |
| P9 | 0.3761 | 1.504 | 75.2 | 0.196 | 0.928 | 92.8 | 84.0 | 12.4 | 14.8 |
| P10 | 0.3564 | 1.411 | 70.5 | 0.1761 | 0.815 | 81.5 | 76.0 | 7.8 | 10.2 |
| P11 | 0.3142 | 1.210 | 60.5 | 0.1672 | 0.765 | 76.5 | 68.5 | 11.3 | 16.5 |
| P12 | 0.203 | 0.682 | 34.1 | 0.1267 | 0.537 | 53.7 | 43.9 | 13.8 | 31.5 |
| P13 | 0.2972 | 1.130 | 56.5 | 0.3238 | 1.649 | 164.9 | 110.7 | 76.7 | 69.3 |
| P14 | 0.292 | 1.105 | 55.2 | 0.163 | 0.742 | 74.2 | 64.7 | 13.4 | 20.7 |
| P15 | 0.3006 | 1.146 | 57.3 | 0.1316 | 0.564 | 56.4 | 56.9 | 0.6 | 1.1 |
| P16 | 0.2172 | 0.750 | 37.5 | 0.1103 | 0.444 | 44.4 | 41.0 | 4.9 | 12.0 |
| P17 | 0.2614 | 0.960 | 48.0 | 0.132 | 0.567 | 56.7 | 52.3 | 6.1 | 11.7 |
| P18 | 0.6862 | 2.977 | 148.8 | 0.196 | 0.928 | 92.8 | 120.8 | 39.6 | 32.8 |
| P19 | 0.4766 | 1.981 | 99.1 | 0.2809 | 1.407 | 140.7 | 119.9 | 29.4 | 24.5 |
| P20 | 0.266 | 0.981 | 49.1 | 0.133 | 0.572 | 57.2 | 53.1 | 5.8 | 10.8 |
| P21 | 0.4155 | 1.691 | 84.6 | 0.1588 | 0.718 | 71.8 | 78.2 | 9.0 | 11.6 |
| P22 | 0.2194 | 0.760 | 38.0 | 0.1192 | 0.494 | 49.4 | 43.7 | 8.1 | 18.5 |
| P23 | 0.3518 | 1.389 | 69.4 | 0.1719 | 0.792 | 79.2 | 74.3 | 6.9 | 9.3 |
| P24 | 0.1618 | 0.487 | 24.3 | 0.1164 | 0.479 | 47.9 | 36.1 | 16.6 | 46.1 |
| P25 | 0.4853 | 2.023 | 101.1 | 0.1966 | 0.931 | 93.1 | 97.1 | 5.7 | 5.8 |
| P26 | 0.2566 | 0.937 | 46.8 | 0.1429 | 0.628 | 62.8 | 54.8 | 11.3 | 20.6 |
| P27 | 0.2271 | 0.797 | 39.8 | 0.1409 | 0.617 | 61.7 | 50.8 | 15.4 | 30.4 |
| P28 | 0.4942 | 2.065 | 103.3 | 0.2651 | 1.318 | 131.8 | 117.5 | 20.2 | 17.2 |
| P29 | 0.247 | 0.891 | 44.6 | 0.1172 | 0.483 | 48.3 | 46.4 | 2.6 | 5.7 |
| P30 | 0.5732 | 2.440 | 122.0 | 0.2569 | 1.271 | 127.1 | 124.6 | 3.6 | 2.9 |
| P31 | 0.5598 | 2.377 | 118.8 | 0.1516 | 0.677 | 67.7 | 93.3 | 36.1 | 38.7 |
| P32 | 0.2997 | 1.142 | 57.1 | 0.2394 | 1.173 | 117.3 | 87.2 | 42.6 | 48.8 |
| P33 | 0.2344 | 0.831 | 41.6 | 0.1229 | 0.515 | 51.5 | 46.5 | 7.0 | 15.1 |
| P34 | 0.2536 | 0.923 | 46.1 | 0.1473 | 0.653 | 65.3 | 55.7 | 13.6 | 24.3 |
| P35 | 0.2037 | 0.686 | 34.3 | 0.179 | 0.832 | 83.2 | 58.7 | 34.6 | 58.9 |
| P36 | 0.2619 | 0.962 | 48.1 | 0.1329 | 0.572 | 57.2 | 52.6 | 6.4 | 12.2 |
| N1 | 0.2353 | 0.836 | 41.8 | 0.1605 | 0.727 | 72.7 | 57.3 | 21.9 | 38.2 |
| N2 | 0.3675 | 1.463 | 73.2 | 0.1766 | 0.818 | 81.8 | 77.5 | 6.1 | 7.9 |
| N3 | 0.1691 | 0.521 | 26.1 | 0.1216 | 0.508 | 50.8 | 38.4 | 17.5 | 45.5 |
| N4 | 0.2021 | 0.678 | 33.9 | 0.1133 | 0.461 | 46.1 | 40.0 | 8.6 | 21.6 |
| N5 | 0.1322 | 0.346 | 17.3 | 0.0896 | 0.327 | 32.7 | 25.0 | 10.9 | 43.6 |
| N6 | 0.1333 | 0.351 | 17.6 | 0.0962 | 0.365 | 36.5 | 27.0 | 13.4 | 49.4 |
| N7 | 0.2012 | 0.674 | 33.7 | 0.2125 | 1.021 | 102.1 | 67.9 | 48.4 | 71.2 |
| N8 | 0.1508 | 0.434 | 21.7 | 0.1434 | 0.631 | 63.1 | 42.4 | 29.3 | 69.0 |
| N9 | 0.2002 | 0.669 | 33.5 | 0.1225 | 0.513 | 51.3 | 42.4 | 12.6 | 29.8 |
| N10 | 0.2328 | 0.824 | 41.2 | 0.1574 | 0.710 | 71.0 | 56.1 | 21.1 | 37.6 |
| N11 | 0.1606 | 0.481 | 24.1 | 0.1014 | 0.394 | 39.4 | 31.7 | 10.8 | 34.2 |
| N12 | 0.1431 | 0.398 | 19.9 | 0.0884 | 0.321 | 32.1 | 26.0 | 8.6 | 33.1 |
| N13 | 0.1879 | 0.611 | 30.5 | 0.1202 | 0.500 | 50.0 | 40.3 | 13.8 | 34.2 |
| N14 | 0.1805 | 0.575 | 28.8 | 0.1418 | 0.622 | 62.2 | 45.5 | 23.6 | 51.9 |
| N15 | 0.2624 | 0.964 | 48.2 | 0.2048 | 0.977 | 97.7 | 73.0 | 35.0 | 48.0 |
| N16 | 0.1399 | 0.383 | 19.1 | 0.1334 | 0.574 | 57.4 | 38.3 | 27.1 | 70.7 |
| N17 | 0.1636 | 0.495 | 24.8 | 0.0916 | 0.339 | 33.9 | 29.3 | 6.4 | 21.9 |
| N18 | 0.128 | 0.326 | 16.3 | 0.0893 | 0.326 | 32.6 | 24.4 | 11.5 | 47.0 |
| N19 | 0.1579 | 0.468 | 23.4 | 0.1207 | 0.503 | 50.3 | 36.8 | 19.0 | 51.6 |
| N20 | 0.1366 | 0.367 | 18.4 | 0.1345 | 0.581 | 58.1 | 38.2 | 28.1 | 73.5 |
| N21 | 0.1778 | 0.563 | 28.1 | 0.1559 | 0.701 | 70.1 | 49.1 | 29.7 | 60.5 |
| N22 | 0.207 | 0.701 | 35.1 | 0.128 | 0.544 | 54.4 | 44.7 | 13.7 | 30.6 |
| N23 | 0.1146 | 0.263 | 13.1 | 0.1021 | 0.398 | 39.8 | 26.5 | 18.8 | 71.2 |
| N24 | 0.1684 | 0.518 | 25.9 | 0.0973 | 0.371 | 37.1 | 31.5 | 7.9 | 25.1 |
| N25 | 0.1878 | 0.610 | 30.5 | 0.1168 | 0.481 | 48.1 | 39.3 | 12.4 | 31.6 |
| N26 | 0.4144 | 1.686 | 84.3 | 0.1741 | 0.804 | 80.4 | 82.4 | 2.8 | 3.3 |
| N27 | 0.1526 | 0.443 | 22.2 | 0.1146 | 0.468 | 46.8 | 34.5 | 17.5 | 50.6 |
| N28 | 0.1629 | 0.492 | 24.6 | 0.1038 | 0.407 | 40.7 | 32.7 | 11.4 | 35.0 |
| N29 | 0.1568 | 0.463 | 23.1 | 0.1066 | 0.423 | 42.3 | 32.7 | 13.6 | 41.4 |
| N30 | 0.1589 | 0.473 | 23.6 | 0.1033 | 0.405 | 40.5 | 32.1 | 11.9 | 37.1 |
| N31 | 0.2193 | 0.760 | 38.0 | 0.1517 | 0.678 | 67.8 | 52.9 | 21.1 | 39.8 |
| N32 | 0.1625 | 0.490 | 24.5 | 0.1477 | 0.655 | 65.5 | 45.0 | 29.0 | 64.4 |
| N33 | 0.1556 | 0.457 | 22.9 | 0.1086 | 0.435 | 43.5 | 33.2 | 14.6 | 43.9 |
| N34 | 0.139 | 0.378 | 18.9 | 0.2389 | 1.170 | 117.0 | 68.0 | 69.3 | 102.0 |
| N35 | 0.1225 | 0.300 | 15.0 | 0.1142 | 0.466 | 46.6 | 30.8 | 22.4 | 72.5 |
| N36 | 0.2205 | 0.765 | 38.3 | 0.0886 | 0.322 | 32.2 | 35.2 | 4.3 | 12.3 |

TABLE 6
Serum ENT concentrations determined using standard curve derived from test set (5 & 6)

| Set 3 | Rep 5-OD | | Serum | Rep 6-OD | | Serum | | | |
|---|---|---|---|---|---|---|---|---|---|
| Sample | (dilution | Calculated | level | (dilution | Calculated | level | Mean | STD | CV |
| Name | 20X) | (ng/ml) | (ng/ml) | 50X) | (ng/ml) | (ng/ml) | (ng/ml) | (ng/ml) | (%) |
| P1 | 0.4573 | 3.844 | 76.9 | 0.1511 | 0.966 | 48.3 | 62.6 | 20.2 | 32.3 |
| P2 | 0.4341 | 3.626 | 72.5 | 0.1425 | 0.885 | 44.3 | 58.4 | 20.0 | 34.2 |
| P3 | 0.372 | 3.042 | 60.8 | 0.1436 | 0.896 | 44.8 | 52.8 | 11.4 | 21.5 |
| P4 | 0.7347 | 6.451 | 129.0 | 0.2966 | 2.334 | 116.7 | 122.9 | 8.7 | 7.1 |
| P5 | 0.2653 | 2.039 | 40.8 | 0.1221 | 0.694 | 34.7 | 37.7 | 4.3 | 11.4 |
| P6 | 0.6229 | 5.400 | 108.0 | 0.1853 | 1.288 | 64.4 | 86.2 | 30.8 | 35.8 |
| P7 | 0.3648 | 2.975 | 59.5 | 0.115 | 0.627 | 31.3 | 45.4 | 19.9 | 43.8 |
| P8 | 0.4567 | 3.838 | 76.8 | 0.1548 | 1.001 | 50.0 | 63.4 | 18.9 | 29.8 |
| P9 | 0.3583 | 2.914 | 58.3 | 0.1356 | 0.820 | 41.0 | 49.6 | 12.2 | 24.6 |
| P10 | 0.3277 | 2.626 | 52.5 | 0.1517 | 0.972 | 48.6 | 50.6 | 2.8 | 5.5 |
| P11 | 0.2878 | 2.251 | 45.0 | 0.134 | 0.805 | 40.3 | 42.6 | 3.4 | 7.9 |
| P12 | 0.2206 | 1.619 | 32.4 | 0.1124 | 0.602 | 30.1 | 31.3 | 1.6 | 5.1 |
| P13 | 0.2195 | 1.609 | 32.2 | 0.1048 | 0.531 | 26.6 | 29.4 | 4.0 | 13.6 |
| P14 | 0.3249 | 2.600 | 52.0 | 0.1494 | 0.950 | 47.5 | 49.8 | 3.2 | 6.4 |
| P15 | 0.3564 | 2.896 | 57.9 | 0.1332 | 0.798 | 39.9 | 48.9 | 12.7 | 26.1 |
| P16 | 0.2028 | 1.452 | 29.0 | 0.1286 | 0.755 | 37.7 | 33.4 | 6.1 | 18.4 |
| P17 | 0.3437 | 2.776 | 55.5 | 0.1395 | 0.857 | 42.9 | 49.2 | 9.0 | 18.2 |
| P18 | 0.5128 | 4.366 | 87.3 | 0.1961 | 1.389 | 69.5 | 78.4 | 12.6 | 16.1 |
| P19 | 0.5972 | 5.159 | 103.2 | 0.1919 | 1.350 | 67.5 | 85.3 | 25.2 | 29.6 |
| P20 | 0.2671 | 2.056 | 41.1 | 0.118 | 0.655 | 32.8 | 36.9 | 5.9 | 16.0 |
| P21 | 0.3242 | 2.593 | 51.9 | 0.1252 | 0.723 | 36.1 | 44.0 | 11.1 | 25.3 |
| P22 | 0.2127 | 1.545 | 30.9 | 0.1227 | 0.699 | 35.0 | 32.9 | 2.9 | 8.7 |
| P23 | 0.4333 | 3.618 | 72.4 | 0.185 | 1.285 | 64.2 | 68.3 | 5.7 | 8.4 |
| P24 | 0.2195 | 1.609 | 32.2 | 0.1523 | 0.977 | 48.9 | 40.5 | 11.8 | 29.1 |
| P25 | 0.5345 | 4.570 | 91.4 | 0.1773 | 1.212 | 60.6 | 76.0 | 21.8 | 28.6 |
| P26 | 0.2607 | 1.996 | 39.9 | 0.125 | 0.721 | 36.0 | 38.0 | 2.7 | 7.2 |
| P27 | 0.2433 | 1.833 | 36.7 | 0.121 | 0.683 | 34.2 | 35.4 | 1.8 | 5.0 |
| P28 | 0.455 | 3.822 | 76.4 | 0.2963 | 2.331 | 116.5 | 96.5 | 28.4 | 29.4 |
| P29 | 0.271 | 2.093 | 41.9 | 0.112 | 0.599 | 29.9 | 35.9 | 8.4 | 23.5 |
| P30 | 0.7163 | 6.278 | 125.6 | 0.2529 | 1.923 | 96.1 | 110.9 | 20.8 | 18.8 |
| P31 | 0.2841 | 2.216 | 44.3 | 0.1243 | 0.714 | 35.7 | 40.0 | 6.1 | 15.2 |
| P32 | 0.2642 | 2.029 | 40.6 | 0.1286 | 0.755 | 37.7 | 39.2 | 2.0 | 5.1 |
| P33 | 0.185 | 1.285 | 25.7 | 0.16 | 1.050 | 52.5 | 39.1 | 18.9 | 48.5 |
| P34 | 0.3259 | 2.609 | 52.2 | 0.1476 | 0.933 | 46.7 | 49.4 | 3.9 | 7.9 |
| P35 | 0.263 | 2.018 | 40.4 | 0.1145 | 0.622 | 31.1 | 35.7 | 6.5 | 18.3 |
| P36 | 0.3252 | 2.602 | 52.0 | 0.1444 | 0.903 | 45.2 | 48.6 | 4.9 | 10.0 |
| N1 | 0.1609 | 1.746 | 34.9 | 0.102 | 1.059 | 53.0 | 43.9 | 12.8 | 29.0 |
| N2 | 0.1249 | 1.326 | 26.5 | 0.0891 | 0.909 | 45.5 | 36.0 | 13.4 | 37.2 |
| N3 | 0.1054 | 1.099 | 22.0 | 0.0888 | 0.906 | 45.3 | 33.6 | 16.5 | 49.0 |
| N4 | 0.1041 | 1.084 | 21.7 | 0.0934 | 0.959 | 48.0 | 34.8 | 18.6 | 53.4 |
| N5 | 0.0967 | 0.998 | 20.0 | 0.0876 | 0.892 | 44.6 | 32.3 | 17.4 | 54.0 |
| N6 | 0.1021 | 1.061 | 21.2 | 0.0766 | 0.763 | 38.2 | 29.7 | 12.0 | 40.4 |
| N7 | 0.1064 | 1.111 | 22.2 | 0.0908 | 0.929 | 46.4 | 34.3 | 17.1 | 49.9 |
| N8 | 0.1446 | 1.556 | 31.1 | 0.0877 | 0.893 | 44.6 | 37.9 | 9.6 | 25.2 |
| N9 | 0.0985 | 1.019 | 20.4 | 0.0798 | 0.801 | 40.0 | 30.2 | 13.9 | 46.0 |
| N10 | 0.105 | 1.094 | 21.9 | 0.2456 | 2.733 | 136.7 | 79.3 | 81.2 | 102.4 |
| N11 | 0.1094 | 1.146 | 22.9 | 0.0845 | 0.855 | 42.8 | 32.8 | 14.0 | 42.8 |
| N12 | 0.1081 | 1.131 | 22.6 | 0.0807 | 0.811 | 40.6 | 31.6 | 12.7 | 40.2 |
| N13 | 0.1155 | 1.217 | 24.3 | 0.0959 | 0.988 | 49.4 | 36.9 | 17.7 | 48.1 |
| N14 | 0.1127 | 1.184 | 23.7 | 0.0893 | 0.911 | 45.6 | 34.6 | 15.5 | 44.7 |
| N15 | 0.1016 | 1.055 | 21.1 | 0.0824 | 0.831 | 41.6 | 31.3 | 14.5 | 46.2 |
| N16 | 0.096 | 0.990 | 19.8 | 0.0798 | 0.801 | 40.0 | 29.9 | 14.3 | 47.9 |
| N17 | 0.1209 | 1.280 | 25.6 | 0.0898 | 0.917 | 45.9 | 35.7 | 14.3 | 40.1 |
| N18 | 0.1036 | 1.078 | 21.6 | 0.087 | 0.885 | 44.2 | 32.9 | 16.0 | 48.7 |
| N19 | 0.1434 | 1.542 | 30.8 | 0.1172 | 1.237 | 61.8 | 46.3 | 21.9 | 47.3 |
| N20 | 0.1097 | 1.149 | 23.0 | 0.0942 | 0.969 | 48.4 | 35.7 | 18.0 | 50.4 |
| N21 | 0.1375 | 1.473 | 29.5 | 0.1676 | 1.824 | 91.2 | 60.3 | 43.7 | 72.4 |
| N22 | 0.141 | 1.514 | 30.3 | 0.0925 | 0.949 | 47.4 | 38.9 | 12.1 | 31.2 |
| N23 | 0.1265 | 1.345 | 26.9 | 0.0861 | 0.874 | 43.7 | 35.3 | 11.9 | 33.7 |
| N24 | 0.1879 | 2.061 | 41.2 | 0.1003 | 1.040 | 52.0 | 46.6 | 7.6 | 16.3 |
| N25 | 0.1593 | 1.727 | 34.5 | 0.0969 | 1.000 | 50.0 | 42.3 | 10.9 | 25.9 |
| N26 | 0.3912 | 4.430 | 88.6 | 0.1899 | 2.084 | 104.2 | 96.4 | 11.0 | 11.4 |
| N27 | 0.1266 | 1.346 | 26.9 | 0.1231 | 1.305 | 65.3 | 46.1 | 27.1 | 58.8 |
| N28 | 0.1427 | 1.534 | 30.7 | 0.0841 | 0.851 | 42.5 | 36.6 | 8.4 | 22.9 |
| N29 | 0.1607 | 1.744 | 34.9 | 0.0949 | 0.977 | 48.8 | 41.9 | 9.9 | 23.6 |
| N30 | 0.1364 | 1.460 | 29.2 | 0.0929 | 0.953 | 47.7 | 38.4 | 13.1 | 34.0 |
| N31 | 0.1758 | 1.920 | 38.4 | 0.1336 | 1.428 | 71.4 | 54.9 | 23.3 | 42.5 |
| N32 | 0.1354 | 1.449 | 29.0 | 0.1125 | 1.182 | 59.1 | 44.0 | 21.3 | 48.4 |
| N33 | 0.1378 | 1.477 | 29.5 | 0.1191 | 1.259 | 62.9 | 46.2 | 23.6 | 51.1 |
| N34 | 0.1136 | 1.195 | 23.9 | 0.0943 | 0.970 | 48.5 | 36.2 | 17.4 | 48.1 |
| N35 | 0.1146 | 1.206 | 24.1 | 0.0865 | 0.879 | 43.9 | 34.0 | 14.0 | 41.2 |
| N36 | 0.109 | 1.141 | 22.8 | 0.078 | 0.780 | 39.0 | 30.9 | 11.4 | 37.0 |

TABLE 7
Assay variation of serum concentrations in breast
cancer patients and health control sample set

| Sample | Endotrophin concentrations in serum samples (ng/ml) | | | | | | | |
|---|---|---|---|---|---|---|---|---|
| Name | Rep 1 | Rep 2 | Rep 3 | Rep 4 | Rep 5 | Rep 6 | Mean | STD |
| P1 | 155.5 | 67.6 | 54.0 | 76.3 | 76.9 | 48.3 | 79.8 | 38.9 |
| P2 | 171.7 | 133.2 | 42.0 | 71.2 | 72.5 | 44.3 | 89.2 | 52.1 |
| P3 | 165.1 | 115.7 | 44.0 | 58.7 | 60.8 | 44.8 | 81.5 | 48.7 |
| P5 | 123.9 | 80.5 | 36.6 | 49.2 | 40.8 | 34.7 | 61.0 | 35.1 |
| P6 | 247.8 | 181.9 | 78.4 | 83.4 | 108.0 | 64.4 | 127.3 | 72.3 |
| P7 | 204.9 | 113.0 | 82.2 | 86.3 | 59.5 | 31.3 | 96.2 | 59.9 |
| P8 | 281.3 | 184.4 | 61.8 | 80.9 | 76.8 | 50.0 | 122.5 | 91.4 |
| P9 | 223.9 | 164.6 | 75.2 | 92.8 | 58.3 | 41.0 | 109.3 | 70.6 |
| P10 | 303.3 | 149.0 | 70.5 | 81.5 | 52.5 | 48.6 | 117.6 | 98.0 |
| P12 | 171.2 | 76.6 | 34.1 | 53.7 | 32.4 | 30.1 | 66.4 | 54.3 |
| P13 | 180.0 | 99.3 | 56.5 | 164.9 | 32.2 | 26.6 | 93.3 | 66.7 |
| P14 | 162.0 | 76.2 | 55.2 | 74.2 | 52.0 | 47.5 | 77.9 | 42.9 |
| P15 | 190.3 | 107.8 | 57.3 | 56.4 | 57.9 | 39.9 | 84.9 | 56.5 |
| P16 | 105.2 | 77.6 | 37.5 | 44.4 | 29.0 | 37.7 | 55.2 | 29.7 |
| P17 | 162.2 | 97.8 | 48.0 | 56.7 | 55.5 | 42.9 | 77.2 | 46.0 |
| P18 | 282.1 | 240.7 | 148.8 | 92.8 | 87.3 | 69.5 | 153.5 | 88.6 |
| P20 | 120.5 | 86.1 | 49.1 | 57.2 | 41.1 | 32.8 | 64.5 | 33.0 |
| P21 | 249.9 | 152.0 | 84.6 | 71.8 | 51.9 | 36.1 | 107.7 | 80.3 |
| P22 | 224.5 | 129.5 | 38.0 | 49.4 | 30.9 | 35.0 | 84.6 | 77.9 |
| P23 | 272.1 | 161.0 | 69.4 | 79.2 | 72.4 | 64.2 | 119.7 | 83.0 |
| P24 | 141.4 | 86.5 | 24.3 | 47.9 | 32.2 | 48.9 | 63.5 | 43.7 |
| P25 | 155.7 | 143.0 | 101.1 | 93.1 | 91.4 | 60.6 | 107.5 | 35.5 |
| P26 | 94.7 | 74.7 | 46.8 | 62.8 | 39.9 | 36.0 | 59.2 | 22.7 |
| P27 | 106.8 | 74.3 | 39.8 | 61.7 | 36.7 | 34.2 | 58.9 | 28.3 |
| P28 | 304.0 | 164.5 | 103.3 | 131.8 | 76.4 | 116.5 | 149.4 | 81.2 |
| P29 | 92.2 | 69.5 | 44.6 | 48.3 | 41.9 | 29.9 | 54.4 | 22.6 |
| P30 | 355.6 | 247.3 | 122.0 | 127.1 | 125.6 | 96.1 | 179.0 | 101.5 |
| P31 | 143.7 | 133.0 | 118.8 | 67.7 | 44.3 | 35.7 | 90.5 | 47.1 |
| P32 | 147.8 | 117.0 | 57.1 | 117.3 | 40.6 | 37.7 | 86.3 | 46.9 |
| P33 | 98.6 | 56.8 | 41.6 | 51.5 | 25.7 | 52.5 | 54.5 | 24.3 |
| P34 | 142.8 | 69.3 | 46.1 | 65.3 | 52.2 | 46.7 | 70.4 | 36.7 |
| P35 | 137.7 | 45.8 | 34.3 | 83.2 | 40.4 | 31.1 | 62.1 | 41.5 |
| P36 | 131.5 | 65.9 | 48.1 | 57.2 | 52.0 | 45.2 | 66.7 | 32.6 |
| N1 | 58.8 | 41.6 | 41.8 | 72.7 | 34.9 | 53.0 | 50.5 | 13.9 |
| N2 | 74.2 | 55.0 | 73.2 | 81.8 | 26.5 | 45.5 | 59.4 | 21.0 |
| N3 | 67.6 | 32.6 | 26.1 | 50.8 | 22.0 | 45.3 | 40.7 | 17.2 |
| N4 | 75.7 | 42.4 | 33.9 | 46.1 | 21.7 | 48.0 | 44.6 | 18.0 |
| N5 | 42.1 | 15.4 | 17.3 | 32.7 | 20.0 | 44.6 | 28.7 | 12.9 |
| N6 | 78.0 | 24.9 | 17.6 | 36.5 | 21.2 | 38.2 | 36.1 | 22.1 |
| N7 | 53.0 | 24.1 | 33.7 | 102.1 | 22.2 | 46.4 | 46.9 | 29.6 |
| N8 | 92.5 | 75.2 | 21.7 | 63.1 | 31.1 | 44.6 | 54.7 | 27.1 |
| N9 | 62.4 | 37.2 | 33.5 | 51.3 | 20.4 | 40.0 | 40.8 | 14.6 |
| N10 | 55.2 | 38.2 | 41.2 | 71.0 | 21.9 | 136.7 | 60.7 | 40.8 |
| N11 | 94.0 | 64.0 | 24.1 | 39.4 | 22.9 | 42.8 | 47.9 | 27.1 |
| N12 | 83.5 | 58.5 | 19.9 | 32.1 | 22.6 | 40.6 | 42.9 | 24.3 |
| N13 | 127.5 | 44.5 | 30.5 | 50.0 | 24.3 | 49.4 | 54.4 | 37.3 |
| N14 | 114.6 | 51.8 | 28.8 | 62.2 | 23.7 | 45.6 | 54.5 | 32.8 |
| N15 | 151.2 | 44.5 | 48.2 | 97.7 | 21.1 | 41.6 | 67.4 | 48.3 |
| N16 | 97.9 | 44.6 | 19.1 | 57.4 | 19.8 | 40.0 | 46.5 | 29.2 |
| N17 | 139.2 | 68.2 | 24.8 | 33.9 | 25.6 | 45.9 | 56.3 | 43.7 |
| N18 | 336.0 | 44.2 | 16.3 | 32.6 | 21.6 | 44.2 | 82.5 | 124.7 |
| N20 | 134.9 | 43.4 | 18.4 | 58.1 | 23.0 | 48.4 | 54.4 | 42.3 |
| N21 | 186.1 | 41.0 | 28.1 | 70.1 | 29.5 | 91.2 | 74.3 | 60.1 |
| N22 | 192.5 | 56.5 | 35.1 | 54.4 | 30.3 | 47.4 | 69.4 | 61.2 |
| N23 | 222.5 | 14.3 | 13.1 | 39.8 | 26.9 | 43.7 | 60.1 | 80.6 |
| N24 | 124.0 | 59.0 | 25.9 | 37.1 | 41.2 | 52.0 | 56.5 | 35.0 |
| N25 | 62.1 | 35.8 | 30.5 | 48.1 | 34.5 | 50.0 | 43.5 | 12.0 |
| N26 | 278.7 | 178.9 | 84.3 | 80.4 | 88.6 | 104.2 | 135.9 | 79.0 |
| N27 | 77.7 | 53.1 | 22.2 | 46.8 | 26.9 | 65.3 | 48.7 | 21.5 |
| N28 | 80.7 | 36.3 | 24.6 | 40.7 | 30.7 | 42.5 | 42.6 | 19.8 |
| N29 | 78.1 | 45.8 | 23.1 | 42.3 | 34.9 | 48.8 | 45.5 | 18.4 |
| N30 | 64.9 | 41.4 | 23.6 | 40.5 | 29.2 | 47.7 | 41.2 | 14.5 |
| N31 | 101.4 | 50.0 | 38.0 | 67.8 | 38.4 | 71.4 | 61.2 | 24.3 |
| N32 | 86.5 | 47.0 | 24.5 | 65.5 | 29.0 | 59.1 | 51.9 | 23.4 |
| N33 | 69.5 | 47.5 | 22.9 | 43.5 | 29.5 | 62.9 | 46.0 | 18.2 |
| N34 | 73.3 | 37.5 | 18.9 | 117.0 | 23.9 | 48.5 | 53.2 | 36.8 |
| N35 | 56.1 | 30.8 | 15.0 | 46.6 | 24.1 | 43.9 | 36.1 | 15.4 |
| N36 | 55.6 | 26.8 | 38.3 | 32.2 | 22.8 | 39.0 | 35.8 | 11.6 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, NY, 1988.
Abbondanzo et al., Am. J. Pediatr. Hematol. Oncol. 12(4): 480-489, 1990.
Allred et al., Arch. Surg. 125(1), 107-113: 1990.
Atherton et al., Biol. of Reproduction 32: 155-171, 1985.
Barzon et al., Euro Surveill. 11: 21(32), 2016.
Beltramello et al., Cell Host Microbe 8: 271-283, 2010.
Brown et al., J. Immunol. Meth. 130(1): 111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm. 74(2):4 25-433, 1977.
De Jager et al., Semin. Nucl. Med. 23(2): 165-179, 1993.
Dholakia et al., J. Biol. Chem. 264: 20638-20642, 1989.
Diamond et al., J. Virol. 77: 2578-2586, 2003.
Doolittle and Ben-Zeev, Methods Mol. Biol. 109: 215-237, 1999.
Duffy et al., N. Engl. J. Med. 360: 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet. 3: 231-236, 1977.
Gornet et al., Semin Reprod Med. 34(5): 285-292, 2016.
Gulbis and Galand, Hum. Pathol. 24(12): 1271-1285, 1993.
Halfon et al., PLoS ONE 5(5): e10569, 2010
Hessell et al., Nature 449: 101-104, 2007.
Khatoon et al., Ann. of Neurology 26: 210-219, 1989.
King et al., J. Biol. Chem. 269: 10210-10218, 1989.
Kohler and Milstein, Eur. J. Immunol. 6: 511-519, 1976.
Kohler and Milstein, Nature 256: 495-497, 1975.
Kyte and Doolittle, J. Mol. Biol. 157(1): 105-132, 1982.
Mansuy et al., Lancet Infect Dis. 16(10): 1106-7, 2016.
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Sun et al., J. Clin. Invest. 121:2094, 2011.
Sun et al., Cell Metabol. 18:470, 2013.
Sun et al., Nature Comm., 5:3485, 2014.
Sun et al., Diabetologia 60:24-29, 2017.
Tang et al., J. Biol. Chem., 271: 28324-28330, 1996.
Wawrzynczak & Thorpe, In: Immunoconjugates, Antibody Conuugates In Radioimaging And Therapy Of Cancer, Vogel (Ed.), NY, Oxford University Press, 28, 1987.
Yu et al., J Immunol Methods 336: 142-151, 2008.

1. A method of detecting human endotrophin in a sample comprising:
(a) providing a surface coated with ETN-4.1;
(b) contacting the coated surface with a sample;
(c) washing the coated surface;
(d) contacting the coated surface with a solution of ETN-1Rb;
(e) washing the coated surface;
(f) contacting the coated surface with a labeled antibody that binds selectively to ETN-1Rb;
(g) washing the coated surface; and
(h) determined labeled antibody binding to ETN-1Rb on the coated surface wherein a binding of the labeled antibody to the surface indicates the present of endotrophin in the sample.

2. The method of claim 1, wherein washing steps (c), (e) and/or (g) are repeated prior to the following step.

3. The method of claim 1, further comprising treating the surface of step (a) with a buffered blocking solution comprising an inert protein prior to step (b).

4. The method of claim 3, wherein the inert protein is albumin, such as bovine serum albumin, optionally at 3% wt/vol concentration.

5. The method of claim 1, wherein the solution of ETN-1Rb of step (d) comprises an inert protein.

6. The method of claim 5, wherein the inert protein is albumin, such as bovine serum albumin, optionally at 3% wt/vol concentration.

7. The method of claim 1, further comprising a step, prior to step (a), of coating the surface with a solution of ETN-4.1 to produce the coated surface of step (a).

8. The method of claim 1, wherein the labeled antibody is labeled with a ligand, an enzyme, a chromophore, a fluorophore, a dye, or a radiolabel.

9. The method of claim 8, wherein the enzyme is horseradish peroxidase.

10. The method of claim 1, further comprising performing a positive control reaction and/or a negative control reaction and/or a standard curve reaction.

11. The method of claim 3, wherein treating the surface of step (a) with a buffered blocking solution is performed for about an hour.

12. The method of claim 7, wherein coating the surface with a solution of ETN-4.1 to produce the coated surface of step (a) is performed for about 12-20 hours, such as about 16 hours.

13. The method of claim 1, wherein step (f) is performed for about 1-2 hours.

14. The method of claim 1, wherein step (d) is performed for about an hour.

15. The method of claim 1, wherein the sample is whole blood, plasma, serum, or tissue extract.

16. A kit comprising:
(a) antibody ETN4.1;
(b) antibody ETN-1Rb; and
(c) a labeled antibody that binds selectively to ETN-1R.

17. The kit of claim 16, wherein said ETN4.1u is disposed on a surface, such as the surface of a well, a plate, or membrane.

18. The kit of claim 16, further comprising one or more buffered solutions or a reagent for making the same.

19. The kit of claim 16, further comprising one or more reagents for detection of the labeled antibody.

20. The kit of claim 16, further comprising reagents for performing a positive and/or negative control reaction for binding of ETN4.1, ETN-1Rb and/or the labeled antibody.
